(19) United States
(12) Patent Application Publication
(10) Pub. No.: US 2012/0282209 A1
(43) Pub. Date: Nov. 8, 2012

(54) MULTI-PHASE HAIR CONDITIONER

(76) Inventors: Tanapatr Barameesangpet, Lardkrabang (TH); Busarin Kasemchainan, Ladkrabang (TH)

(21) Appl. No.: 13/512,898

(22) PCT Filed: Dec. 3, 2010
(86) PCT No.: PCT/EP2010/068829

§ 371 Date: Jul. 19, 2012

(30) Foreign Application Priority Data
Dec. 11, 2009 (EP) 09178961.0

(51) Int. Cl. A61K 8/73; A61Q 5/12

(52) U.S. Cl. 424/70.13

(57) ABSTRACT

Packaged multi-phase hair conditioner comprising at least two visibly distinct phases, said composition comprising a nonionic surfactant and a water-swellable starch.

The present invention relates to a multiphase conditioning composition.

In a first aspect the present invention relates to a packaged multi-phase hair conditioner comprising at least two visibly distinct phases, said composition comprising a cationic surfactant and a water-swellable starch.

The composition according to the invention presents visually distinct phases on extrusion immediately prior to use.

The visually distinct phases may be in contact in the package or they may be separated. Examples of packaging which separates distinct phases which are extruded together include tubes with internal webs, side by side tube arrangements and tube-in-tube arrangements. Examples of packaging where at least some of the phases are in contact with one another inside the packaging are standard tubes whereby the distinct phases are co-extruded into the package in a pattern which reflects the pattern seen when the composition is extruded by the consumer immediately prior to use and the co-called shoulder-striping mechanisms used by striped toothpaste tubes.

In a preferred embodiment at least one of the distinct phases is in contact with at least one other phase inside the packaging employed. More preferably, the arrangement of the phases inside the packaging employed reflects the arrangement seen when the composition is dispensed by the user immediately prior to use.

Preferably, the composition has a viscosity of from 80 Pa·s to 200 Pa·s, preferably from 90 to 110 Pa·s as measured on a Brookfield viscometer at 30 C, 60 sec, cps (Brookfield RVT, Spindle TC, Speed 10 rpm, Helipath stand, 150 ml standard glass beaker).

This ensures that the arrangement of the phases inside the package is reasonably maintained when the product is gradually dispensed by the user.

Preferably, the water swellable starch is non-ionic.

Preferably, the water swellable starch is derived from maize.

Preferably, the water swellable starch is a stabilized and cross-linked.

Preferably, the water swellable starch is agglomerated pregelatinised starch.

Preferably, the water swellable starch is hydroxypropylated.

Suitable water swellable starches include Structure XL® ex. Akzo Nobel.

Preferably, the composition is packaged in a deformable tube. The tube will comprise a dispensing assembly which will comprise an exit orifice. Preferably, the exit orifice will have a diameter of from 7 to 10 mm so that the compositional arrangement of the visually distinct phases is maintained after extrusion immediately prior to use.

The visually distinct phases can be arranged in any pattern. Typical patterns include side by side, coaxial, striped and quartered. Preferably, the composition comprises two distinct phases which, when viewed in section transverse to the direction of extrusion from the package, are quartered. By quartered is meant that the face is cut into four sections by two transverse lines and the diagonally opposite sections, or quarters, are similarly coloured to each other and differently coloured to the other pair of quarters.

Preferably, the composition comprises two distinct phases each of which may be present in from 1 to 4, preferably 2 portions.

Preferably, the phases are present in equal measures.

Surprisingly, the composition provides better manageability in dry hair.

The composition according to the invention comprises one or more conditioning surfactants which are cosmetically acceptable and suitable for topical application to the hair.

Suitable conditioning surfactants are selected from cationic surfactants, used singly or in admixture. Examples include quaternary ammonium hydroxides or salts thereof, e.g. chlorides.

Suitable cationic surfactants for use in hair conditioners of the invention include cetyltrimethylammonium chloride, behenyltrimethylammonium chloride, cetylpyridinium chloride, tetramethylammonium chloride, tetraethylammonium chloride, octyltrimethylammonium chloride, dodecyltrimethylammonium chloride, hexadecyltrimethylammonium chloride, octyldimethylbenzylammonium chloride, decyldimethylbenzylammonium chloride, stearyldimethylbenzylammonium chloride, didodecyldimethylammonium chloride, dioctadecyldimethylammonium chloride, tallowtrimethylammonium chloride, cocotrimethylammonium chloride, PEG-2 oleylammonium chloride and the corresponding hydroxides thereof. Further suitable cationic surfactants include those materials having the CTFA designations Quaternium-5, Quaternium-31 and Quaternium-18. Mixtures of any of the foregoing materials may also be suitable. A particularly useful cationic surfactant for use in hair conditioners of the invention is cetyltrimethylammonium chloride, available commercially, for example as GENAMIN CTAC, ex Hoechst Celanese.

Salts of primary, secondary, and tertiary fatty amines are also suitable cationic surfactants. The alkyl groups of such amines preferably have from about 12 to about 22 carbon atoms, and can be substituted or unsubstituted.

Particularly useful are amido substituted tertiary fatty amines. Such amines, useful herein, include stearamidopropyldimethylamine, stearamidopropyidiethylamine, stearamidoethyldiethylamine, stearamidoethyldimethylamine, palmitamidopropyld imethylamine, palmitamidopropyldiethylamine, palmitamidoethyldiethylamine, palmitamidoethyldimethylamine, behenamidopropyldimethylamine, behenamidopropyldiethylamine, behenamidoethyldiethylamine, behenamidoethyldimethylamine, arachidamidopropyldimethylamine, arachidamidopropyldiethylamine, arachidamidoethyldiethylamine, arachidamidoethyldimethylamine, diethylaminoethylstearamide. Also useful are dimethylstearamine, dimethylsoyamine, soyamine, myristylamine, tridecylamine, ethylstearylamine, N-tallowpropane diamine, ethoxylated (with 5 moles of ethylene oxide) stearylamine, dihydroxyethylstearylamine, and arachidyl behenylamine. These amines are typically used in combination with an acid to provide the cationic species. The preferred acid useful herein includes L-glutamic acid, lactic acid, hydrochloric acid, malic acid, succinic acid, acetic acid, fumaric acid, tartaric acid, citric acid, L-glutamic hydrochloride, and mixtures thereof; more preferably L-glutamic acid, lactic acid, citric acid. Cationic amine surfactants included among those useful in the present invention are disclosed in U.S. Pat. No. 4,275,055 to Nachtigal, et al., issued Jun. 23, 1981.

The molar ratio of protonatable amines to H⁺from the acid is preferably from about 1:0.3 to 1:1.2, and more preferably from about 1:0.5 to about 1:1.1.

In conditioners of the invention, the level of cationic surfactant is preferably from 0.01 to 10, more preferably 0.05 to 5, most preferably 0.1 to 2 wt % of the total composition.

The conditioning composition may comprise any of a number of ingredients commonly found in conditioning compositions.

Conditioners of the invention preferably incorporate a fatty alcohol material. The combined use of fatty alcohol materials and cationic surfactants in conditioning compositions is believed to be especially advantageous, because this leads to the formation of a lamellar phase, in which the cationic surfactant is dispersed.

Representative fatty alcohols comprise from 8 to 22 carbon atoms, more preferably 16 to 20. Examples of suitable fatty alcohols include cetyl alcohol, stearyl alcohol and mixtures thereof. The use of these materials is also advantageous in that they contribute to the overall conditioning properties of compositions of the invention.

The level of fatty alcohol material in conditioners of the invention is conveniently from 0.01 to 10, preferably from 0.1 to 5 wt % by weight of the total composition. The weight ratio of cationic surfactant to fatty alcohol is suitably from 10:1 to 1:10, preferably from 4:1 to 1:8, optimally from 1:1 to 1:4.

Preferably, the composition comprises silicones. Silicones which impart a conditioning benefit to the hair are well known in the art. Suitable silicones have an average particle size of from 0.01 to 1 micron.

Compositions of this invention may contain any other ingredient normally used in hair treatment formulations. These other ingredients may include viscosity modifiers, preservatives, colouring agents, polyols such as glycerine and polypropylene glycol, chelating agents such as EDTA, antioxidants, fragrances, antimicrobials and sunscreens. Each of these ingredients will be present in an amount effective to accomplish its purpose. Generally these optional ingredients are included individually at a level of up to 5 wt % of the total composition.

Preferably, compositions of this invention also contain adjuvants suitable for hair care. Generally such ingredients are included individually at a level of up to 2, preferably up to 1 wt % of the total composition.

Among suitable hair care adjuvants, are:

(i) natural hair root nutrients, such as amino acids and sugars. Examples of suitable amino acids include arginine, cysteine, glutamine, glutamic acid, isoleucine, leucine, methionine, serine and valine, and/or precursors and derivatives thereof. The amino acids may be added singly, in mixtures, or in the form of peptides, e.g. di- and tripeptides. The amino acids may also be added in the form of a protein hydrolysate, such as a keratin or collagen hydrolysate. Suitable sugars are glucose, dextrose and fructose. These may be added singly or in the form of, e.g. fruit extracts. A particularly preferred combination of natural hair root nutrients for inclusion in compositions of the invention is isoleucine and glucose. A particularly preferred amino acid nutrient is arginine.

(ii) hair fibre benefit agents. Examples are:
ceramides, for moisturising the fibre and maintaining cuticle integrity. Ceramides are available by extraction from natural sources, or as synthetic ceramides and pseudoceramides. A preferred ceramide is Ceramide II, ex Quest. Mixtures of ceramides may also be suitable, such as Ceramides LS, ex Laboratoires Serobiologiques.

Preferably, the composition comprises less than 5% wt. anionic surfactant. More preferably the composition comprises less than 3% wt. anionic surfactant and most preferably less than 1% wt. anionic surfactant.

The invention will now be described with reference to the following non-limiting examples.

### EXAMPLE 1

The following is a multi-phase conditioning composition.

| | Phase I - White | Phase II-Colour |
|---|---|---|
| | phase | phase |
| | (% as is) | (% as is) |
| Water | To 100 | To 100 |
| Cetearyl Alcohol | 7.0000 | 7.0000 |
| Behentrimonium Chloride | 4.3500 | 4.3500 |
| Dimethicone and C12-15 | 1.6700 | 1.6700 |
| Pareth-3 and C12-15 Pareth-23 | | |
| Hydroxypropyl Starch | 1.0000 | 1.0000 |
| Phosphate* | | |
| Perfume | 0.7500 | 0.7500 |
| Disodium EDTA | 0.1000 | 0.1000 |
| Preservative | 0.1000 | 0.1000 |
| Preservative | 0.0600 | 0.0600 |
| Colourant | 0.1000 | |
| Colourant | | 0.015 |
| Colourant | | 0.01 |

*Structure XL ex. Akzo Nobel.

### EXAMPLE 2

The composition according to example 1 is made by the following process. Add hot water (75-80° C.) in the main mixer at medium speed.

In a side mixer prepare the fat and heat to 85° C. to melt with maximum mixing speed. Then add the cationic surfactant and maintain the temperature at 82-85° C. with stirring until a clear solution is provided.

Add the contents of the side mixer to the main mixer and flush with hot (75° C.) water.

Maintain the temperature of the main mixer at 75-80° C. with turn on the homogenizer to form emulsion for 10-15 min. and then start cooling the jacket to 66-68° C. with stirring at medium speed.

Then charge the chill water (18-25° C.) into the main mixer with high speed agitator to ensure the homogenous between base with water.

In a side mixer add water then EDTA and stir at medium speed for around 3 minutes until EDTA dissolved. Add the Structure XL with stirring at high speed for 10 minutes checking for no lumps. The transfer to main mixer and flush with hot (75° C.) water.

With the temperature of the main mixer at less than 45° C. add the silicone, perfume, preservatives and buffers in the usual manner.

1. Packaged multi-phase hair conditioner comprising at least two visibly distinct phases, said composition comprising a cationic surfactant and a water-swellable starch.

2. Composition according to claim 1 having a viscosity of from 80 000 to 200 000 centipoise as measured on a Brookfield viscometer at 30° C., 60 sec measured on a Brookfield RVT, Spindle TC, Speed 10 rpm, Helipath stand, 150 ml standard glass beaker.

3. Composition according to claim 1 wherein the water swellable starch has a nonionic charge.

4. Composition according to claim 1 wherein the water swellable starch is a modified agglomerated pregelatinsed maize starch.

5. Composition according to claim 1 wherein the composition is packaged in a deformable tube.

6. Composition according to claim 1 comprising two distinct phases which, when viewed in section transverse to the direction of extrusion from the package, are quartered.
